(19) United States
(12) Patent Application Publication
(10) Pub. No.: US 2009/0149762 A1
(43) Pub. Date: Jun. 11, 2009

(54) Apparatus for displaying the blood pressure value and method thereof

(76) Inventors: Hsing Ou Yang, Zhonghe City (TW); Hsin-Lung Tu, Xinzhuang City (TW); Feng Shuo Chang, Luzhu Shiang (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/320,080

(22) Filed: Jan. 16, 2009

Related U.S. Application Data
(63) Continuation-in-part of application No. 11/445,171, filed on Jun. 2, 2006.

(51) Int. Cl. A61B 5/02

(52) U.S. Cl. 600/485

(57) ABSTRACT

An apparatus for displaying the blood pressure and a method thereof are disclosed. The apparatus uses a blood-measuring unit for receiving an analog blood pressure signal; a photoplethysmography (PPG) signal measuring unit for receiving an analog PPG signal; an analog-to-digital converter for converting the analog blood pressure signal into a digital blood pressure signal and the analog PPG signal into a digital PPG signal; an input unit for generating a human data; a microprocessor unit for receiving the digital blood pressure signal, the digital PPG signal and the human data so as to generate a different value in response to the digital blood pressure signal and the digital PPG signal, and output a pulse wave velocity (PWV) signal in response to the operation of the different value and the human data; an LCD driving unit for outputting an first LCD driving voltage and a second LCD driving voltage respectively in response to the digital blood pressure signal and the pulse wave velocity (PWV) signal; an LCD unit for displaying the colors that correspond to the first LCD driving and the second LCD driving voltage respectively.

### REFERENCE TO RELATED APPLICATION

This application is a Continuation-in-Part of application Ser. No. 11/445,171 filed on 2 Jun. 2006, and entitled AN APPARATUS FOR DISPLAYING BLOOD PRESSURE VALUE AND A METHOD THEREOF.

### BACKGROUND OF THE INVENTION

1. Field of the Invention

The present invention relates to an apparatus for displaying blood pressure and a method thereof. In particular, this invention relates to an apparatus for displaying the blood pressure and a method thereof that uses an emitting light of an LCD unit to display the status of measured blood pressure.

2. Description of the Related Art

Blood pressure is the pressure generated in blood vessels when blood circulates through a body. When the heart is in systole, blood is transmitted to the entire body via aortas. The generated pressure is named systolic pressure. When the heart is in diastole, blood in the body flows back to the heart. The generated pressure is named diastolic pressure. When blood pressure is abnormal, the blood pressure is an indictor of diseases, such as apoplexy, heart disease, or uremia, etc. Some patients don't feel uncomfortable when their blood pressure is abnormal. Therefore, measuring the blood pressure gives us an insight into our physical condition.

Blood pressure usually is measured by pressing blood vessels via a tourniquet and stethoscope. Blood-measuring instruments can be divided into two types—mercury sphygmomanometer and auscultation sphygmomanometer. Their common point is the use of a tourniquet filled with air to press the artery so as to stop the flood. In this time, the artery under the tourniquet does not generate a pulse. Then, the air in the tourniquet is relaxed to allow the artery to generate pulses again and the pulse is detected. When the first sound of the Korotoff Sound is detected, the measured value in the pressure gauge is systolic pressure. Then, the air in the tourniquet continues to be released. The channel in the blood vessel becomes larger and the pulsing time of blood in the blood vessel becomes longer. Until the pressure in the tourniquet is less than a predetermined value, the pulsing effect of blood becomes less. When the Korotoff Sound becomes passivated, the final Korotoff Sound means diastolic pressure has been found. The mercury sphygmomanometer has a mercury display for displaying the measured blood pressure. When the mercury column of the mercury display is faulty, the display of the mercury sphygmomanometer doesn't function. Therefore, the auscultation sphygmomanometer was developed. The auscultation sphygmomanometer displays the measured blood pressure on the LCD. It is convenient for the user to obtain their blood pressure. However, the user has to refer to a normal blood pressure table to obtain the status of the blood pressure. The user cannot directly obtain their own blood pressure status. It is inconvenient.

Additionally, photoplethysmography (PPG) is an electro-opitc technique to measure the pulse wave of vessels. The measuring apparatus for PPG is the pulse oximeter, which considers the relative absorption of Hemoglobin and Oxyhemoglobin to non-invasive measure of arterial oxygen saturation (SpO2) using the dual-wavelength illumination (LED), and the signal measured by PPG is called photoplethysmographic signal.

The arterial oxygen saturation (SpO2) PPG is a fundamental parameter to diagnose the heart and lung function, the information on the idiosyncrasy in circulating system, the status of heart vessels, and hypoxia, etc.

Especially, PPG signal is one of vital signal, basic signal to measure vital parameters of a case, and therefore PPS is used as an important monitoring aid at casual wards and intensive care units.

### SUMMARY OF THE INVENTION

One particular aspect of the present invention is to provide an apparatus for displaying the blood pressure and a method thereof. The present invention uses the colors emitting from an LCD unit to display the statuses of the measured blood pressure and the vein induration. After the blood pressure is measured, the user obtains the blood pressure status and the vein induration status, without having to refer to a normal blood table and a vein induration table.

The apparatus for displaying the blood pressure includes a blood-measuring unit, a photoplethysmography (PPG) signal measuring unit, an input unit, a microprocessor unit, an analog-to-digital converter, an LCD driving unit and an LCD unit. The blood-measuring unit receives an analog blood pressure signal. The photoplethysmography (PPG) signal measuring unit receives an analog PPG signal. The analog-to-digital converter couples to the blood-measuring unit and the photoplethysmography (PPG) signal measuring unit. The analog-to-digital converter converts the analog blood pressure signal and the analog PPG signal into a digital blood pressure signal and a digital PPG signal respectively. The input unit generates a human data.

The microprocessor unit receives the digital blood pressure signal, the digital PPG signal and the human data for generating a different value in response to the digital blood pressure signal and the digital PPG signal, and outputting a pulse wave velocity (PWV) signal in response to the operation of the different value and the human data. The LCD driving unit outputs a first LCD driving voltage and a second LCD driving voltage in response to digital blood pressure signal and the pulse wave velocity (PWV) signal respectively. The LCD unit couples to the LCD driving unit and is controlled by the first LCD driving voltage to display a color that corresponds to the first LCD driving voltage or controlled by the second LCD driving voltage to display a color that corresponds to the second LCD driving voltage.

The method for displaying blood pressure includes obtaining an analog blood pressure signal, amplifying the analog blood pressure signal, obtaining an analog PPG signal, receiving a human data; converting the analog blood pressure signal and the analog PPG signal into a digital blood pressure signal and a digital PPG signal respectively, generating a first LCD driving voltage according to the digital blood pressure signal, and displaying a light with a color that corresponds to the first LCD driving voltage. Moreover, the method for displaying blood pressure further includes computing for generating a different value in response to the digital blood pressure signal and the digital PPG signal, computing for generating a pulse wave velocity (PWV) signal in response to the different value and the human data, generating a second LCD driving voltage that corresponds to the pulse wave velocity (PWV) signal according to the pulse wave velocity (PWV) signal; and displaying a light with a color that corresponds to the second LCD driving voltage.

For further understanding of the invention, reference is made to the following detailed description illustrating the embodiments and examples of the invention. The description is only for illustrating the invention and is not intended to be considered limiting of the scope of the claim.

### BRIEF DESCRIPTION OF THE DRAWINGS

The drawings included herein provide a further understanding of the invention. A brief introduction of the drawings is as follows:

FIG. 1 is a schematic diagram of the appearance of the apparatus for displaying the blood pressure of the present invention;

FIG. 2 is a schematic diagram of the applications of the present invention;

FIG. 3 is a block diagram of the apparatus for displaying the blood pressure of the present invention;

FIG. 4 is a flow chart of the method for displaying the blood pressure of the present invention; and

FIGS. 5A-5B are the schematic diagrams of the wave form of the present invention.

### DETAILED DESCRIPTION OF THE PREFERRED EMBODIMENTS

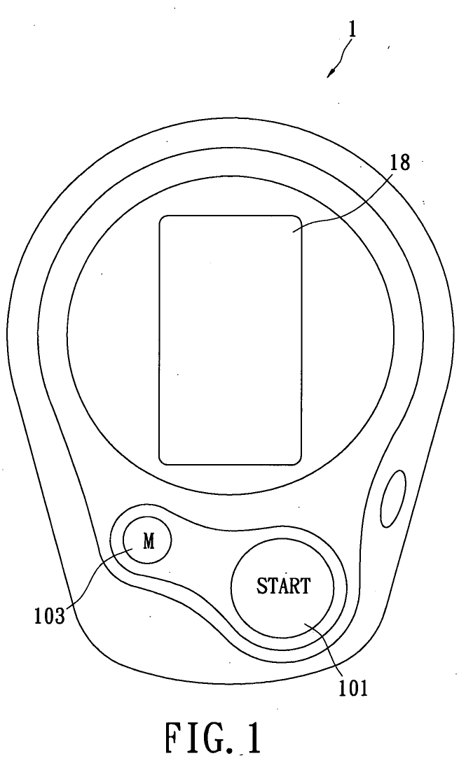

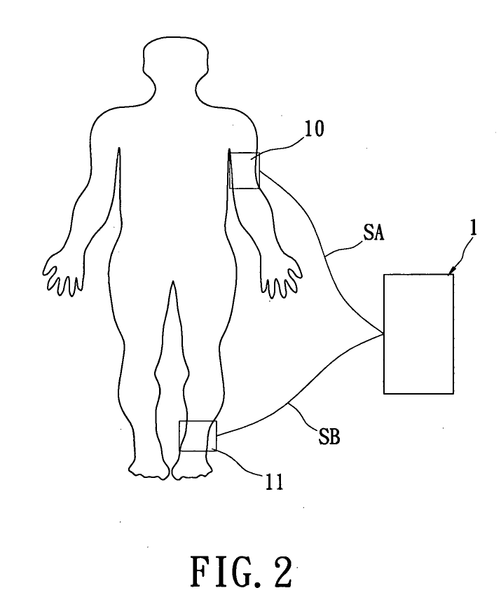

Reference is made to FIG. 1 and FIG. 2, the FIG. 1 shows a schematic diagram of the appearance of the apparatus for displaying the blood pressure of the present invention and the FIG. 2 shows a schematic diagram of the applications of the present invention. On the apparatus for displaying the blood pressure 1, there is an LCD unit 18, a blood pressure measuring button 101, and a blood pressure recording button 103. There are other units in the apparatus for displaying the blood pressure 1. The apparatus for displaying the blood pressure 1 obtains an analog blood pressure signal SA via a blood pressure measuring unit 10 (including an air tube connected with a tourniquet) and obtains an analog PPG signal SB via a photoplethysmography (PPG) signal measuring unit 11 (including another air tube connected with another tourniquet). The user presses the blood pressure measuring button 101 to measure the blood pressure and the pulsation of blood vessels, and presses the blood pressure recording button 103 to store the blood pressure and the pulsation of blood vessels, when the blood pressure and the pulsation of blood vessels are measured.

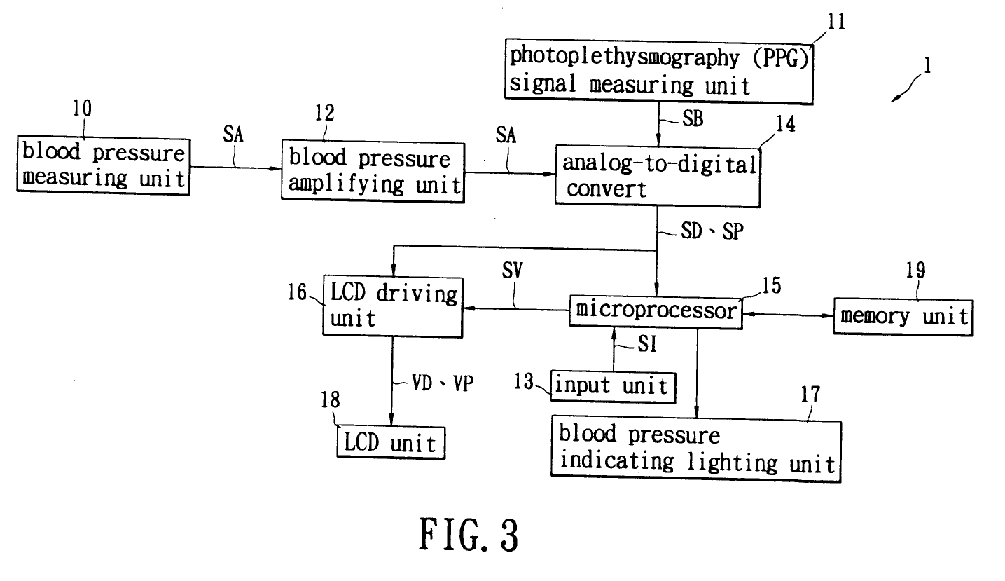

Reference is made to FIG. 3, which shows a block diagram of the apparatus for displaying the blood pressure of the present invention. The apparatus for displaying the blood pressure 1 includes a blood-measuring unit 10, a photoplethysmography (PPG) signal measuring unit 11, a blood pressure amplifying unit 12, an input unit 13, an analog-to-digital converter 14, an LCD driving unit 16, and an LCD unit 18. The apparatus for displaying the blood pressure 1 uses the blood-measuring unit 10 that is composed of an air tube (not shown in the figure) connected with a tourniquet (not shown in the figure) to receive an analog blood pressure signal SA. Moreover, the blood pressure 1 further uses the photoplethysmography (PPG) signal measuring unit 11 that is composed of another air tube (not shown in the figure) connected with another tourniquet (not shown in the figure) to receive an analog PPG signal SB.

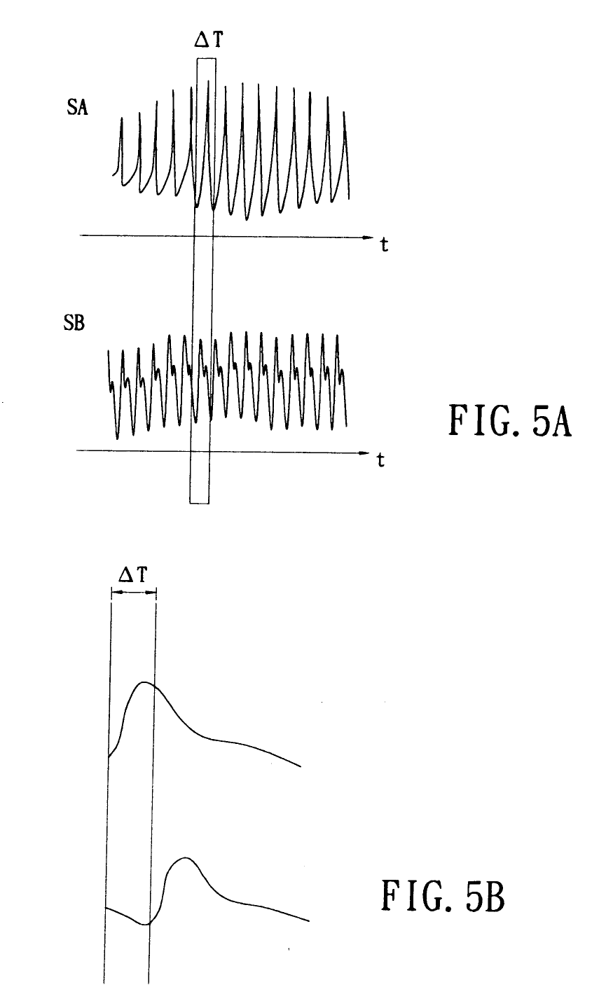

Reference is made to FIGS. 5A-5B, the symbol ΔT is represented to the different time for sampling a difference value (SA−SB) of the analog blood pressure signal SA and the analog PPG signal SB.

The analog blood pressure signal SA is amplified by the blood pressure amplifying unit 12 that is connected with the blood-measuring unit 10 and is then transmitted to the analog-to-digital converter 14 that couples to the blood pressure amplifying unit 12. The analog-to-digital converter 14 converts the analog blood pressure signal SA and the analog PPG signal SB into a digital blood pressure signal SD and a digital PPG signal SP respectively. The LCD driving unit 16 couples to the analog-to-digital converter 14. The LCD driving unit 16 receives the digital blood pressure signal SD so as to output a first LCD driving voltage VD. The LCD unit 18 couples to the LCD driving unit 16 and is controlled by the first LCD driving voltage VD to display a color that corresponds to the first LCD driving voltage VD. The LCD unit 18 can display a backlight with various colors, including red, yellow, and green, etc.

Reference is made to FIG. 3. The apparatus for displaying the blood pressure 1 further includes a microprocessor unit 15. The microprocessor unit 15 couples to the input unit 13, the analog-to-digital converter 14 and the LCD driving unit 16. The microprocessor unit 15 receives the digital blood pressure signal SD and the digital PPG signal SP from the analog-to-digital converter 14, and receives a human data SI from the input unit 13.

Furthermore, the microprocessor unit 15 computes for generating the different value in response to the digital blood pressure signal SD and the digital PPG signal SP, and computes for generating a pulse wave velocity (PWV) signal SV in response to the different value and the human data SI. Wherein the pulse wave velocity (PWV) signal SV can be shown as equation (1),

$$SV = \frac{SA - SB}{Δ T} \qquad ( 1 )$$

Reference is made to FIG. 3 again. The LCD driving unit 16 couples to the microprocessor unit 15 so as to receive the pulse wave velocity (PWV) signal SV and output a second LCD driving voltage VP in response to the pulse wave velocity (PWV) signal SV. The LCD unit 18 couples to the LCD driving unit 16 and is controlled by the second LCD driving voltage VP to display a color that corresponds to the second LCD driving voltage VP. The LCD unit 18 can display a backlight with various colors, including red, yellow, and green, etc.

The microprocessor unit 15 further couples to a blood pressure indicating lighting unit 17. The microprocessor unit 15 controls the blood pressure indicating lighting unit 17 to shine according to the digital blood pressure signal SD. The blood pressure indicating lighting unit 17 is composed of at least one LED. The color of the LED can be red, yellow, or green. The microprocessor unit 15 further couples to a memory unit 19. The memory unit 19 is used for storing the digital blood pressure signal SD.

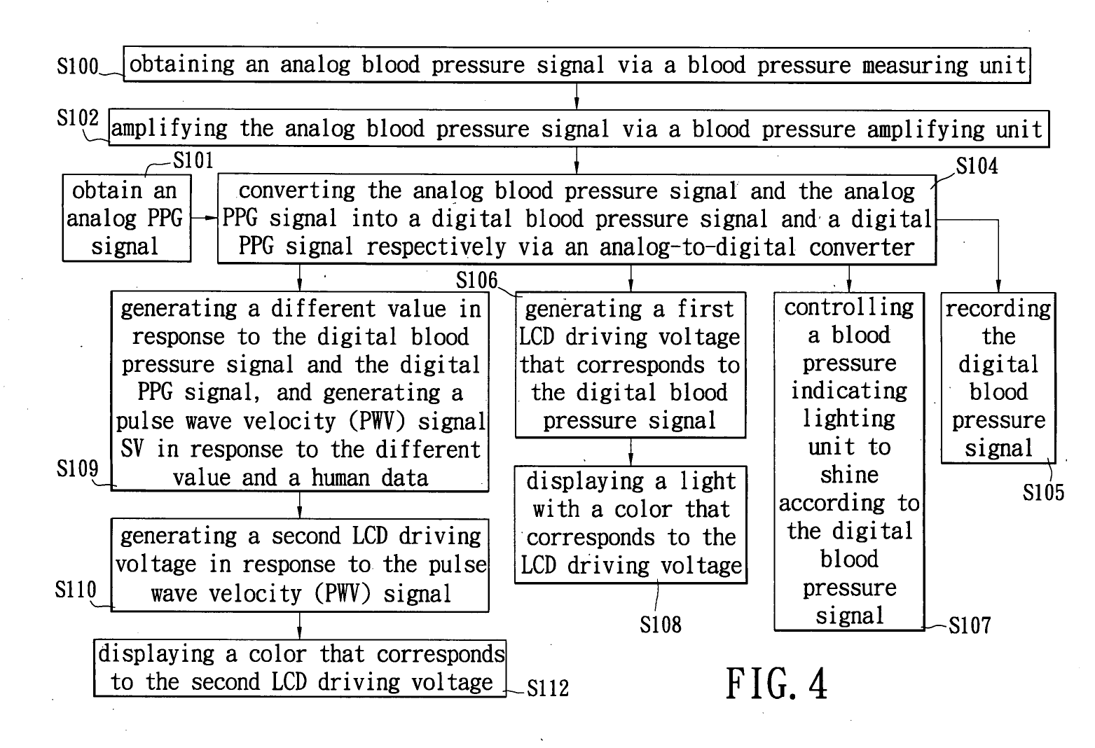

Reference is made to FIG. 3 and FIG. 4. FIG. 4 shows a flow chart of the method for displaying the blood pressure of the present invention. When the user uses the apparatus for displaying the blood pressure to measure the blood measure, the user uses the blood-measuring unit 10 that is composed of an air tube (not shown in the figure) connected with a tourniquet (not shown in the figure) to obtain an analog blood pressure signal SA (S100). Furthermore, the user further uses the photoplethysmography (PPG) signal measuring unit 11 that is composed of another air tube (not shown in the figure) connected with another tourniquet (not shown in the figure) to obtain an analog PPG signal SB (S101).

The measured analog blood pressure signal SA is amplified by the blood pressure amplifying unit 12 (S102) and is then transmitted to the analog-to-digital converter 14. The analog-to-digital converter 14 converts the analog blood pressure signal SA and the analog PPG signal SB into a digital blood pressure signal SD and a digital PPG signal SB respectively (S104), and transmits the digital blood pressure signal SD and the digital PPG signal SB to the LCD driving unit 16 and the microprocessor 15. The LCD driving unit 16 generates and outputs the corresponding first LCD driving voltage VD according to the digital blood pressure signal SD (S106). The microprocessor unit 15 controls the blood pressure indicating lighting unit 17 to shine (S107) according to the digital blood pressure signal SD, and stores the digital blood pressure signal SD in the memory unit (S105). The first LCD driving voltage VD is used for controlling the LCD unit 18 to display a light with a corresponding color (S108).

Moreover, the microprocessor unit 15 computes for generating a different value in response to the digital blood pressure signal SD and the digital PPG signal SP, and computes for generating a pulse wave velocity (PWV) signal SV in response to the different value and the human data SI (S109). Furthermore the LCD driving unit 16 generates a second LCD driving voltage VP in response to the pulse wave velocity (PWV) signal SV (S110). Then, the LCD unit 18 is controlled by the second LCD driving voltage VP to display a color that corresponds to the second LCD driving voltage VP (S112).

The present invention provides an apparatus for displaying the blood pressure and a method thereof. The present invention uses the colors emitting from the LCD unit 18 to show the statuses of the measured blood pressure and the vein induration. After the blood pressure is measured, the user obtains the blood pressure status and the vein induration status, and records the measured blood pressure and the vein induration value. The user easily obtains their health condition without having to refer to a normal blood table.

The description above only illustrates specific embodiments and examples of the invention. The invention should therefore cover various modifications and variations made to the herein-described structure and operations of the invention, provided they fall within the scope of the invention as defined in the following appended claims.

What is claimed is:

1. An apparatus for displaying the blood pressure, comprising:
a blood-measuring unit for receiving an analog blood pressure signal;
a photoplethysmography (PPG) signal measuring unit for receiving an analog PPG signal;
an analog-to-digital converter coupled to the blood-measuring unit and the photoplethysmography (PPG) signal measuring unit, wherein the analog-to-digital converter converts the analog blood pressure signal into a digital blood pressure signal and converts the analog PPG signal into a digital PPG signal;
an input unit for generating a human data;
a microprocessor unit coupled to the analog-to-digital converter and the input unit, wherein the microprocessor unit receives the digital blood pressure signal, the digital PPG signal and the human data for generating a different value in response to the digital blood pressure signal and the digital PPG signal, and outputting a pulse wave velocity (PWV) signal in response to the operation of the different value and the human data;
an LCD driving unit coupled to the analog-to-digital converter and the microprocessor unit, wherein the LCD driving unit receives the digital blood pressure signal and the pulse wave velocity (PWV) signal so as to output a first LCD driving voltage in response to the digital blood pressure signal and output a second LCD driving voltage in response to the pulse wave velocity (PWV) signal; and
an LCD unit coupled to the LCD driving unit and controlled by the first LCD driving voltage to display the color that corresponds to the first LCD driving voltage and controlled by the second LCD driving voltage to display the color that corresponds to the second LCD driving voltage.

2. The apparatus for displaying the blood pressure as claimed in claim 1, wherein the human data is a body height data, an age data and a sex data.

3. The apparatus for displaying the blood pressure as claimed in claim 1, wherein the LCD unit displays a backlight with a color, including red, yellow, or green.

4. The apparatus for displaying the blood pressure as claimed in claim 1, further comprising a blood pressure amplifying unit coupled to the blood-measuring unit and analog-to-digital converter, wherein the blood pressure amplifying unit amplifies the analog, blood pressure signal and then transmits the analog blood pressure signal to the analog-to-digital converter.

5. The apparatus for displaying the blood pressure as claimed in claim 4, wherein the microprocessor further couples to a blood pressure indicating lighting unit, and the microprocessor unit controls the blood pressure indicating lighting unit to shine according to the digital blood pressure signal.

6. The apparatus for displaying the blood pressure as claimed in claim 5, wherein the blood pressure indicating lighting unit is composed of at least one LED.

7. The apparatus for displaying the blood pressure as claimed in claim 6, wherein the color of the LED is red, yellow, green, or white.

8. The apparatus for displaying the blood pressure as claimed in claim 5, wherein the microprocessor further couples to a memory unit for storing the digital blood pressure signal.

9. A method for displaying the blood pressure, comprising:
obtaining an analog blood pressure signal;
amplifying the analog blood pressure signal;
obtaining an analog PPG signal;
receiving a human data;
converting the analog blood pressure signal and the analog PPG signal into a digital blood pressure signal and a digital PPG signal respectively;
generating a first LCD driving voltage that corresponds to the digital blood pressure signal according to the digital blood pressure signal; and
displaying a light with a color that corresponds to the first LCD driving voltage;
computing for generating a different value in response to the digital blood pressure signal and the digital PPG signal;
computing for generating a pulse wave velocity (PWV) signal in response to the different value and the human data;
generating a second LCD driving voltage that corresponds to the pulse wave velocity (PWV) signal according to the pulse wave velocity (PWV) signal; and
displaying a light with a color that corresponds to the second LCD driving voltage.

10. The method for displaying the blood pressure as claimed in claim 9, wherein after the step of converting the analog blood pressure signal into a digital blood pressure signal, the method further comprises a step of controlling a blood pressure indicating lighting unit to shine according to the digital blood pressure signal.

11. The method for displaying the blood pressure as claimed in claim 9, wherein after the step of converting the analog blood pressure signal into a digital blood pressure signal, the method further comprises a step of recording the digital blood pressure signal.
